(19) United States
(12) Patent Application Publication
(10) Pub. No.: US 2008/0220465 A1
(43) Pub. Date: Sep. 11, 2008

(54) Antibiotic Sensitivity Testing Method

(75) Inventors: Gal Ingber, Oranit (IL); Jonathan Gurfkinkel, Omer (IL)

Correspondence Address: THE WEBB LAW FIRM, P.C., 700 KOPPERS BUILDING, 436 SEVENTH AVENUE, PITTSBURGH, PA, 15219, US

(73) Assignee: Pocared Diagnostics, Inc.

(21) Appl. No.: 12/035,482

(22) Filed: Feb. 22, 2008

Related U.S. Application Data
(60) Provisional application No. 60/904,926, filed on Mar. 5, 2007.

(51) Int. Cl. C12Q 1/18

(52) U.S. Cl. 435/32

(57) ABSTRACT

The method includes several steps including obtaining a bacterial sample; identifying the type of bacteria in the bacterial sample; selecting a set of antibiotics based on the identity of the bacteria in the bacterial sample; obtaining a control sample from the bacterial sample; placing the bacterial sample in solutions containing the set of antibiotics; determining concentration of bacteria in the respective antibiotic solutions; determining growth curves for the respective antibiotic solutions based on the determined bacterial concentration; and comparing the growth curves for the respective antibiotic solutions with a growth curve determined from the control sample. An identification and quantification system may be used to select the set of antibiotics, and further may be used in the steps of determining concentration of bacteria in the respective antibiotic solutions and determining growth curves for the respective antibiotic solutions based on the determined bacterial concentration.

### CROSS REFERENCE TO RELATED APPLICATION

This application claims the benefit of U.S. Provisional Application No. 60/904,926 filed Mar. 5, 2007 and entitled “Rapid Antibiotics Sensitivity Testing”.

### BACKGROUND OF THE INVENTION

1. Field of the Invention

The invention is directed to methods and systems of determining effectiveness of antibiotics on bacterial samples.

2. Description of Related Art

In current practice and instrumentation it can take between 30 and 48 hours to obtain information on the sensitivity of bacteria to a selected set of antibiotics. During this period, a patient is either waiting for a result without treatment or is being treated without specific knowledge regarding the bacterial infection. In either situation, there can be negative and even life-threatening consequences for the patient.

Typically in the medical field there are two major approaches for determining the effectiveness of antibiotics on bacteria. In the first approach, a sample of the suspected bacteria is obtained and a colony of the bacteria is grown. It usually takes at least 24 hours of bacterial growth to achieve a bacterial count that is sufficient for testing purposes. Once this level is reached, the bacterial growth is “plated” with different antibiotics. An additional 24 hours is needed while the effectiveness of the antibiotic is evaluated based on the growth of each individual sample.

A second major approach is to take a colony of the suspected bacteria, once again after waiting at least 24 hours for sufficient bacterial growth to occur. Once a sufficient sample size is obtained, the sample is placed in an evaluation instrument with different antibiotics and the instrument detects the differences in growth between the different samples. This evaluation process typically requires at least 6 hours to determine the effectiveness of the different applied antibiotics. In either of the foregoing processes, there is an initial 24 hour delay for the initial formation of bacterial colonies. After this initial delay, a second delay of at least 6 hours occurs. Moreover, since the identity of the bacteria is still unknown after the first 24 hours, the entire range of relevant antibiotics needs to be tested. Further, as noted previously, in either process a patient is either waiting for a result without treatment or is being treated without specific knowledge regarding the bacterial infection. In either situation, there can be negative and even life-threatening consequences for the patient. Accordingly, there is a need in the medical field for an improved way of testing the effectiveness of antibiotics and, correspondingly, the sensitivity of bacteria to a selected set of antibiotics.

Within the medical field, systems and methods are known that utilize optical measurements to test the sensitivity of antibiotics. Several examples of such devices are discussed hereafter. U.S. Pat. No. 5,573,927 to Nelson discloses a method of determining the effectiveness of an antibiotic against bacteria that includes the steps of displaying Raman spectra of a first set of target cells of an initially cultured bacteria, culturing the target cells of a second set in a growth medium free of antibiotics, displaying the Raman spectra of the cells of the second set prior to mitosis, culturing the target cells of a third set in a growth medium containing an antibiotic, displaying the Raman spectra of the target cells of the third set prior to mitosis and displaying ribosome peaks and comparing the ribosome peaks of the spectra of the second and third sets.

U.S. Pat. No. 5,922,282 to Ledly discloses a system for rapid mycobacterium infection diagnosis and mycobacterium antibiotic sensitivity testing that uses an automated pattern recognition microscope, together with the introduction of the luciferase gene into the mycobacteria tuberculosis by means of a specific plasmid. Then, each transformed bacteria will luminesce and the diagnosis can be made. Next, the luminescing bacteria are challenged by various antibiotics and it is observed whether or not the light is “turned off” (i.e., the bacteria are killed).

U.S. Pat. No. 6,861,230 to Murphy et al. discloses the use of an assay for adenylate kinase in an in-vitro test for the external conditions on the growth characteristics of bacterial cells. The Murphy patent further discloses that such in-vitro tests include tests for the sensitivity of bacteria to an antibiotic.

U.S. Pat. No. 6,140,069 to Wardlaw discloses a method for determining the antibiotic sensitivity of bacteria that includes the steps of providing a microorganism growth medium, an effective amount of target microorganism, and a sensible reagent. The sensible reagent includes the antibiotic to be evaluated and a marker. The sensible reagent is incorporated into the growth medium and the growth medium is inoculated with the target microorganism. Then a growth boundary is determined and the magnitude of the marker is measured at the growth boundary. Finally, the MIC of the antibiotic is measured using the magnitude of the marker signal.

United States Patent Application Publication No. 2005/0095665 to Williams et al. discloses a combined anti-microbial susceptibility and microorganism identification system. The system utilizes a hybrid panel concept in which samples are assayed via fluorescent as well as turbidimetric means and methods. The system also uses a modified clear plastic panel for the simultaneous assay of samples via fluorescent identification and turbidimetric antimicrobial susceptibility testing. United States Patent Application Publication No. 2004/0067547 to Harbron et al. discloses a method for detecting microorganisms and evaluating antimicrobial activity that includes the steps of applying an electric field across a solution containing microorganisms, optically measuring the speed of movement of any microorganism suspended in the solution as a result of the electric field, and identifying the presence of one or more specific microorganisms by comparing the measured values with those of known microorganisms which have been measured under standard conditions.

U.S. Pat. No. 5,112,745 to Lorr discloses a system for the rapid identification of microbial organisms as well as the determination of antibiotic sensitivity by infrared spectroscopy. U.S. Pat. No. 4,448,534 to Wertz et al. discloses a system for determining the minimum inhibitory concentration of drugs and identification of microorganisms.

Other systems and techniques in the medical field involve the use of imaging techniques to determine the susceptibility of bacteria to antibiotics. Several examples of such devices are discussed hereafter. U.S. Pat. Nos. 6,251,624 and 6,153,400, each to Matsumua et al., disclose a method and apparatus for performing microbial antibiotic susceptibility testing. In the Matsumua '624 patent, a method and apparatus are disclosed for performing a plurality of microbiological tests in any order or at the same time. The disclosed feature relates to the use of a number of plates that are placed on or with in a holder of an instrument and incubated in the instrument, in the conventional manner described previously. A transport mechanism for moving plate holders to an imaging device is also disclosed. Moreover, a method is disclosed for detecting and/or enumerating microorganism colonies which require the use of a detection plate including an immobilization layer and a sensor layer.

The Matsumua '400 patent discloses a method for determining the existence and/or degree of resistance of a microorganism to one or more antimicrobial agents and requires the determination of the existence and/or extent of resistance of the microorganism to the antimicrobial agent based on the size of an inhibition zone. A kit for determining the existence and/or degree of resistance of a microorganism to one or more antimicrobial agents and requires a container having a plurality of separate compartments is also disclosed.

U.S. Pat. No. 6,665,429 to Wang discloses a method and associated apparatus for optically scanning a disk carrying an antibiotic agent placed on a nutrient medium on a plate, generating a digitally encoded image of the disk, and electronically processing the digitally encoded image.

U.S. Pat. Nos. 5,726,030 and 5,637,501, each to Ollar et al., disclose methods of automatically testing the sensitivity of a paraffinophilic microorganism to different antimicrobial agents and concentrations thereof. The Ollar '030 patent is directed to a method of automatically testing the sensitivity of a paraffinophilic microorganism to different antimicrobial agents and concentrations thereof and requires the step of placing in each of a plurality of receptacles a slide containing a paraffin coating. The Ollar '501 patent is directed to an apparatus for testing the sensitivity of a paraffinophilic microorganism to different antimicrobial agents and concentrations thereof and requires the use of a plurality of paraffin coated slides.

U.S. Pat. No. 3,780,223 to Perry discloses an antibiotics sensitivity measurement system that measures and displays the size of zones of inhibition which develop around discs of antibiotics on a microbiological diffusion assay.

Other systems and techniques in the medical field are known which do not utilize optical or imaging techniques. Several examples of such devices are known from U.S. Pat. No. 6,750,038 to Nakane; U.S. Pat. No. 6,165,741 to Wilson et al.; U.S. Pat. No. 5,863,754 to Bajard; U.S. Pat. No. 5,789,173 to Peck et al.; U.S. Pat. No. 3,957,583 to Gibson et al.; U.S. Pat. No. 3,773,426 to Mudd. Additional examples may be found in United States Patent Application Publication No. 2005/0048599 to Goldber et al. and International Publication No. WO 95/14105 to Roberts. All of the foregoing patents and published applications are directed to systems and methods for determining antimicrobial or antibiotic sensitivity without optical or imaging techniques. As an example, U.S. Pat. No. 6,165,741 to Wilson et al. discloses a method for detecting the growth of microorganisms that requires the detection of quenching of the phosphorescence by oxygen in a culture medium. U.S. Pat. No. 5,789,173 to Peck et al. discloses a method for rapid antimicrobial susceptibility testing that requires DNA amplification.

Other systems and techniques in the medical field are known for the narrow purpose of detecting and identifying bacteria in a sample. U.S. Pat. No. 4,847,198 to Nelson et al. disclose a method for the identification of bacteria by means of ultra-violet excited resonance Raman spectra. The method includes the steps of contacting a bacterial suspension with a single wavelength in the ultra-violet range. A portion of the light energy used is absorbed and a portion of the light energy is emitted. The emitted light energy is measured and processed to produce spectra which are characteristic of the bacteria. U.S. Pat. No. 5,660,998 to Naumann et al. discloses a method for the detection of microorganisms in a sample which involves the transfer of a region of the surface of a culture carrier to a surface of an optical carrier stamp.

While numerous systems and techniques are known in the medical field for testing the susceptibility of bacteria to certain antibiotics, as evidenced by the foregoing patents and publications, the foregoing systems and techniques operate within the two known templates outlined previously. In these templates, an initial 24 hour growth period is required followed by either a second 24 hour evaluation period or a shortened 6 hour effectiveness evaluation based on determining the relative differences in bacterial growth. Accordingly, even with the foregoing known systems and techniques, a need exists in the medical field for an improved way of testing the effectiveness of antibiotics and, correspondingly, the sensitivity of bacteria to a selected set of antibiotics.

### SUMMARY OF THE INVENTION

In view of the foregoing introduction, methods of determining effectiveness of antibiotics according to several embodiments are described herein. In one embodiment, the method comprises several steps including obtaining a bacterial sample; identifying the type of bacteria in the bacterial sample; selecting a set of antibiotics based on the identity of the bacteria in the bacterial sample; obtaining a control sample from the bacterial sample; placing the bacterial sample in solutions containing the set of antibiotics; monitoring growth of bacteria in the respective antibiotic solutions; and comparing bacterial growth in the respective antibiotic solutions with bacterial growth in the control sample.

Another step in the method may comprise maintaining the control sample under the same conditions as the respective antibiotic solutions. Another step in the method may comprise determining a course of medical treatment based on the comparison results from the step of comparing bacterial growth in the respective antibiotic solutions with bacterial growth in the control sample. The step of placing the bacterial sample in solutions containing the set of antibiotics may comprise mixing the bacterial sample with the respective antibiotic solutions. The step of determining concentration of bacteria in the respective antibiotic solutions may comprise measuring bacterial growth at set time intervals. Measuring bacterial growth at set time intervals may comprise determining concentration of bacteria in the respective antibiotic solutions. Additionally, the method may further comprise determining growth curves for the respective antibiotic solutions based on the bacterial growth measurements. The respective growth curves may be compared with a growth curve determined from the control sample.

An identification and quantification system may be used to select the set of antibiotics. Additionally, the identification and quantification system may be used in the steps of monitoring growth of bacteria in the respective antibiotic solutions and comparing bacterial growth in the respective antibiotic solutions with bacterial growth in the control sample. In one embodiment, the identification and quantification system comprises a computer.

Another embodiment described herein is directed to a method of determining effectiveness of antibiotics, comprising the steps of obtaining a bacterial sample; identifying the type of bacteria in the bacterial sample; selecting a set of antibiotics based on the identity of the bacteria in the bacterial sample; obtaining a control sample from the bacterial sample; placing the bacterial sample in solutions containing the set of antibiotics; determining concentration of bacteria in the respective antibiotic solutions; determining growth curves for the respective antibiotic solutions based on the determined bacterial concentration; and comparing the growth curves for the respective antibiotic solutions with a growth curve determined from the control sample.

In this embodiment, another step may comprise maintaining the control sample under the same conditions as the respective antibiotic solutions. A course of medical treatment may be based on the comparison results of comparing the growth curves for the respective antibiotic solutions with a growth curve determined from the control sample. The step of placing the bacterial sample in solutions containing the set of antibiotics may comprise mixing the bacterial sample with the respective antibiotic solutions.

An identification and quantification system may be used to select the set of antibiotics. The identification and quantification system is desirably used in the determining steps of determining concentration of bacteria in the respective antibiotic solutions and determining growth curves for the respective antibiotic solutions based on the determined bacterial concentration. The identification and quantification system may further be used to compare the growth curves for the respective antibiotic solutions with a growth curve determined from the control sample. As one example, the identification and quantification system may comprise a computer.

Further details and advantages will become clear upon reading the following detailed description in conjunction with the accompanying drawing figure.

### BRIEF DESCRIPTION OF THE DRAWING

FIG. 1 is schematic representation of an antibiotics sensitivity testing method.

### DESCRIPTION OF THE PREFERRED EMBODIMENTS

For purposes of the description hereinafter, spatial orientation terms, if used, shall relate to the referenced embodiment as it is oriented in the accompanying drawing figure or otherwise described in the following detailed description. However, it is to be understood that the embodiments described hereinafter may assume many alternative variations, configurations, and sequences. It is also to be understood that the specific devices, features, and components illustrated in the accompanying drawing figure and described herein are simply exemplary and should not be considered as limiting.

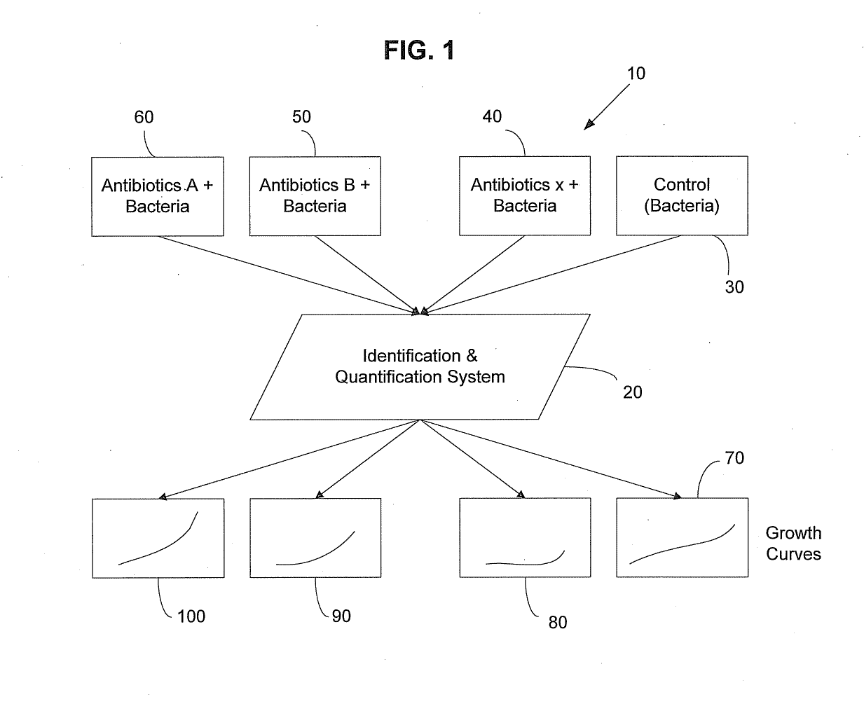

A schematic representation of a system 10 adapted to determine the effectiveness of antibiotics on a bacterial sample is shown in FIG. 1. Generally, the use of system 10 in the determination of the effectiveness of a set, group, or class of antibiotics on a bacterial sample begins with obtaining the bacterial sample. Typically, this step relates to a medical professional, such as doctor, nurse, or clinician taking a bodily fluid sample from a patient. This sample may be placed in a culture or specimen container pursuant to methods customary in the medical field for storage and/or additionally culturing (e.g., growth). A rapid diagnostic system, such as a bacterial identification and quantification system 20, is used to identify the type of bacteria in the bacterial sample container. Identification and quantification system 20 desirably includes a computer platform to perform certain calculations that allow for accomplishment of the methods described in this disclosure. Once identification and quantification system 20 determines the identity of the particular bacterium, system 20 desirably provides a recommendation of a set, class, or grouping of antibiotics which are known to be effective against the identified bacteria. Such antibiotics are designated in FIG. 1 as A, B, through X antibiotics, which may be used to treat the bacteria sample. Identification and quantification system 20 may be preprogrammed with a medical database which may be accessed automatically by an algorithm programmed into the identification and quantification system 20 whereby a set, class, or grouping of selected or recommended antibiotics is suggested to the attending medical professional based on the identity of the bacteria in the bacterial sample. Once a set of antibiotics is selected or identified, it next is desirable to obtain or segregate out a control sample from the bacterial sample and provide the control sample as a solution 30 of the collected bacterial sample.

Next, the bacterial sample is divided into several portions and placed in respective solutions containing antibiotics A-X. As an example, the divided or portioned bacterial sample and antibiotics A-X may be mixed into respective solutions by conventional methods in the medical field and the respective mixed solutions 40, 50, 60 thereby contain mixtures of the collected bacterial sample and the respective antibiotics A-X. Mixed solutions 40-60 are allowed to stand for measurement purposes. Typically, measurement of the respective mixed solutions 40-60 takes the form of monitoring growth of bacteria in the respective mixed solutions containing antibiotics A-X and bacteria sample. The monitoring of bacterial growth is desirably done by identification and quantification system 20 for the respective mixed solutions 40-60. While bacterial growth is occurring in respective mixed solutions 40-60, similar growth is occurring in control sample solution 30 which does not contain an antibiotic. It is desirable according to the instant procedure to maintain control sample solution 30 under the same conditions as the respective mixed 40-60 solutions. Measurement of bacterial growth in all solutions 30-60 is desirably conducted by identification and quantification system 20 at regular or set time intervals.

The ongoing monitoring of growth in solutions 30-60 is accompanied by comparing bacterial growth in the respective antibiotic-containing mixed solutions 40-60 with that occurring in control sample solution 30. Growth curves 70-100 are established for the control sample solution 30, identified as growth curve 70 in FIG. 1, and additionally for the respective antibiotic-containing mixed solutions 40-60, identified as growth curves 80-100 in FIG. 1. These growth curves 70-100 are based or fitted from the measurement of bacterial growth measured by identification and quantification system 20. It is desirable that identification and quantification system 20 comprise devices to monitor bacterial growth and provide this information in electronic form to a computer (not shown) associated with system 20. Such a computer may automatically store bacterial growth information and desirably includes internal algorithms that may estimate and plot growth curves 70-100 from the measured bacterial growth information. Growth curves 70-100 are desirably displayable on a display device, such as a graphical user interface, associated with the computer typically forming part of identification and quantification system 20. Identification and quantification system 20 is further configured and adapted to compare the respective growth curves 80-100 associated with mixed antibiotic and bacterial sample solutions 40-60 with growth curve 70 associated with control sample solution 30. As will be clear from the foregoing, mixed solutions 40-60 and control sample solution 30 are maintained under similar growth conditions and by comparing each growth curve 80-100 with growth curve 70 associated with control sample solution 30 an indication of the relative effectiveness of antibiotics A-X may be determined. This may be done manually, for example, by a medical professional observing the displayed growth curves 70-100 on a computer monitor or GUI or this comparison step may be done automatically by the computer associated with identification and quantification system 20.

In either of the foregoing comparison methods, a decision on medical treatment using antibiotics A-X may be made based on the fitted growth curves 70-100 without having to wait until a set amount of growth has actually occurred in the respective mixed solutions 40-60. In particular, a course of medical treatment involving antibiotics A-X may be made more quickly based on the comparison results of comparing growth curves 80-100 associated with mixed solutions 40-60 with growth curve 70 associated with control sample 30. Accordingly, identifying which antibiotic A-X is most effective on the bacterial sample taken form the patient is more rapidly determined according to the foregoing method when time may be of the essence for the individual patient. Growth curves 70-100 may be graphed or fitted based on bacterial growth information by means customary in the computer field and comparison of growth curves 80-100 with growth curve 70 may be conducted by methods customary in the computer field. The foregoing description may be used by medical professionals to select an appropriate antibiotic class for use on a collected bacterial sample and, further, measure the effect of that antibiotic class on the bacterial sample rapidly using a diagnostic arrangement as depicted in FIG. 1.

While a method for rapidly determining sensitivity of bacteria to antibiotics was described in the foregoing description, those skilled in the art may make modifications and alterations to the disclosed embodiments without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

The invention claimed is:

1. A method of determining effectiveness of antibiotics, comprising the steps of:
(a) obtaining a bacterial sample;
(b) identifying the type of bacteria in the bacterial sample;
(c) selecting a set of antibiotics based on the identity of the bacteria in the bacterial sample;
(d) obtaining a control sample from the bacterial sample;
(e) placing the bacterial sample in solutions containing the set of antibiotics;
(f) monitoring growth of bacteria in the respective antibiotic solutions; and
(g) comparing bacterial growth in the respective antibiotic solutions with bacterial growth in the control sample.

2. A method as claimed in claim 1 further comprising maintaining the control sample under the same conditions as the respective antibiotic solutions.

3. A method as claimed in claim 1 further comprising determining a course of medical treatment based on the comparison results of step (g).

4. A method as claimed in claim 1 wherein step (e) comprises mixing the bacterial sample with the respective antibiotic solutions.

5. A method as claimed in claim 1 wherein step (f) comprises measuring bacterial growth at set time intervals.

6. A method a claimed in claim 5 wherein measuring bacterial growth at set time intervals comprises determining concentration of bacteria in the respective antibiotic solutions.

7. A method as claimed in claim 5 further comprising determining growth curves for the respective antibiotic solutions based on the bacterial growth measurements.

8. A method as claimed in claim 7 further comprising comparing the respective growth curves with a growth curve determined from the control sample.

9. A method as claimed in claim 1 wherein an identification and quantification system is used to select the set of antibiotics in step (c).

10. A method as claimed in claim 1 wherein an identification and quantification system is used in step (f) and step (g).

11. A method as claimed in claim 10 wherein the identification and quantification system comprises a computer.

12. A method of determining effectiveness of antibiotics, comprising the steps of:
(a) obtaining a bacterial sample;
(b) identifying the type of bacteria in the bacterial sample;
(c) selecting a set of antibiotics based on the identity of the bacteria in the bacterial sample;
(d) obtaining a control sample from the bacterial sample;
(e) placing the bacterial sample in solutions containing the set of antibiotics;
(f) determining concentration of bacteria in the respective antibiotic solutions;
(g) determining growth curves for the respective antibiotic solutions based on the bacterial concentration determined from step (f); and
(h) comparing the growth curves for the respective antibiotic solutions with a growth curve determined from the control sample.

13. A method as claimed in claim 12 further comprising maintaining the control sample under the same conditions as the respective antibiotic solutions.

14. A method as claimed in claim 12 further comprising determining a course of medical treatment based on the comparison results of step (h).

15. A method as claimed in claim 12 wherein step (e) comprises mixing the bacterial sample with the respective antibiotic solutions.

16. A method as claimed in claim 12 wherein an identification and quantification system is used to select the set of antibiotics in step (c).

17. A method as claimed in claim 12 wherein an identification and quantification system is used in the determining steps of step (g) and step (h).

18. A method as claimed in claim 17 wherein the identification and quantification system comprises a computer.
